(19) United States
(12) Patent Application Publication
(10) Pub. No.: US 2018/0193075 A1
(43) Pub. Date: Jul. 12, 2018

(54) TECHNIQUE AND METHOD TO LOCALLY DELIVER OBJECTS INTO BONE

(71) Applicant: Revenio Research Oy, Vantaa (FI)

(72) Inventors: Petro MOILANEN, Säynätsalo (FI); Heikki NIEMINEN, Helsinki (FI); Ari SALMI, Helsinki (FI); Edward HAEGGSTRÖM, Helsinki (FI); Alejandro GARCIA PEREZ, Helsinki (FI)

(73) Assignee: Revenio Research Oy, Vantaa (FI)

(21) Appl. No.: 15/917,044

(22) Filed: Mar. 9, 2018

Related U.S. Application Data
(63) Continuation of application No. PCT/FI2015/050589, filed on Sep. 9, 2015.

(51) Int. Cl. A61B 17/88

(52) U.S. Cl. CPC A61B 17/8805; A61B 2017/564; A61B 17/8852

(57) ABSTRACT

An object delivery arrangement is disclosed for delivering objects into bone. The arrangement is configured for generating localized mechanical waves into a tissue, for performing localized deposition of the objects near bone, and for exposing the objects and the bone to said mechanical waves to obtain deposition of the objects into the bone.

### RELATED APPLICATION

This application claims priority as a continuation application under 35 U.S.C. § 120 to PCT/FI2015/050589 filed as an International Application on Sep. 9, 2015 designating the U.S., the entire content of which is hereby incorporated by reference in its entirety.

### FIELD

The present disclosure relates to bone healthcare and health management. Exemplary embodiments deal with detection of a weak bone and healing of the weak or fractured bone in vivo.

### BACKGROUND INFORMATION

Bone diseases are disorders in remodeling of bone tissue. As a result, bones can become mechanically weak. Reduction of bone mineral density (BMD) is a natural process related to aging after the age of 20. However, some bone diseases, such as osteoporosis, can cause excessive loss of BMD. Deficiencies in nutrient intake (e.g., calcium and vitamin D and C), hormonal imbalance and cell abnormalities can also cause bone disorders.

Bone fractures are labelled low-impact fractures and high-impact fractures. The low-impact (or fragility) fractures are predominantly caused by deteriorated bone strength, which results from aging or bone disease, and can occur due to a mechanical impact following for example, slipping or falling. High-impact (or traumatic) fractures require excessive stress caused by traumatic accidents and can occur in healthy bone. Bone is considered weak when the risk for fragility fractures is increased.

There is a need for methods to detect and heal weak bones, preferably before fractures occur.

Localized inference of bone quality techniques are being developed by several research groups. To this end, quantitative ultrasound (QUS) is one of the most promising approaches. Yet, ultrasonic detection of clinically relevant fracture sites such as the hip and vertebrae is challenging and requires further development.

Weak bone is often treated by systemic delivery of drug and growth factors. Such drugs and drug-like factors are absorbed throughout the body. Therefore, high doses may be required to gain sufficient therapeutic effects in the bone. However, the drug, especially at high drug doses, may cause side effects outside fracture sites, some of which may be severe.

Tissue treatment based on localized delivery and release of drugs has been reported for soft tissue sites. For example, a recent report details ultrasound-aided delivery and release in articular cartilage (Nieminen et al., Ultrasound Med Biol 41(8):2259-2268, 2015) and subchondral bone through articular cartilage (Nieminen et al., Ultrasonics Symposium (IUS), 2012 IEEE International, pages 1869-1872). For bone metastases, there are reports on localized ultrasound-aided release of drugs, first transported into the vicinity of the therapy site by blood circulation (Staruch et al., Radiology 263(1):117-127, 2012). However, there is no known method to do simultaneous release and deposition. Moreover, there is no known methodology that would permit construction of a hand-held device for detection of weak bone (site with fracture risk) followed by instant localized treatment.

U.S. Pat. No. 6,231,528 B1 discloses an in vivo technology for using ultrasound in conjunction with a biomedical compound or bone growth factor to induce healing, growth and ingrowth responses in bone. To this end, non-invasively applied ultrasonic stimulus is operative to transport the bone growth factor from the external surface of the soft tissue to the bone and to synergistically enhance the interaction between the bone growth factor and the bone. This technology does not involve deposition of the ultrasonically transported objects and describes the use of ultrasound for delivery only in the context of an extracorporeal ultrasound transducer, an ultrasound pulser, biomedical compounds and bone growth factors. In addition, the technology does not incorporate focused ultrasonic waves which are vital for highly localized treatment.

### SUMMARY

A kit is disclosed, comprising: an object delivery arrangement for delivering objects into bone; and retention means configured to counteract passive diffusion out from a target and formed by one of: a covering layer having a lower perfusion coefficient than embracing tissues; an active object having a size sufficient to prevent passive diffusion out of a target in a bone; a substance that expands and covers a target site in the bone; and an ultrasound, photo-acoustics or plasma source configured to generate localized mechanical waves for maintaining a substance and for subsequently depositing the substance; wherein said arrangement is configured to: perform localized deposition of objects near a bone; expose objects and a bone to localized mechanical waves to force objects into a bone; and perform retention of deposited objects in a bone by using said retention means, so as to prevent deposited objects from escaping a target site in that bone.

### BRIEF DESCRIPTION OF THE DRAWINGS

Other features and advantages disclosed herein will become more apparent from the following detailed description of exemplary embodiments, when read in conjunction with the accompanying drawings wherein the elements are represented by like reference numerals, and wherein:

FIG. 1 shows exemplary embodiments of the present disclosure;

FIG. 2 shows alternate exemplary embodiments of the present disclosure; and

FIG. 3 shows exemplary preliminary results.

### DETAILED DESCRIPTION

An improved technology is disclosed for transport and deposition of objects into bone for effective and controllable localized management of bone health. This can, for example, be achieved by an object delivery arrangement for delivering objects into bone. The arrangement can include for generating localized mechanical waves into a tissue, means for performing localized deposition of objects near bone, and means for exposing the objects and the bone to the mechanical waves to obtain deposition of the objects into the bone.

An object delivery method is disclosed for delivering objects into bone. In the method localized mechanical waves can be generated into a tissue, localized deposition of the objects near bone is performed, and the objects and the bone are exposed to the mechanical waves to obtain deposition of the objects into the bone.

Exemplary embodiments are based on generation of localized mechanical waves into a tissue, and localized deposition of the objects near bone, and deposition of the objects to the bone by the effect of the mechanical waves.

The direction of transportation and deposition of objects into bone tissue is not limited to transport and deposition from bone periosteal (i.e., outer) surface into bone tissue. The transportation and deposition of objects can also be achieved from any surface of a cavity (e.g., endosteal surface) or pore into bone tissue.

An exemplary benefit of embodiments disclosed herein is that the proposed conjunction of means permits an enhanced therapeutic power and advanced management of the therapeutic effect compared to known treatments.

In an exemplary object delivery arrangement for delivering objects into bone as disclosed, the delivery object is, for example, a drug molecule or molecules for osteoporosis treatment. The arrangement can include means for generating localized mechanical waves into a tissue. The means are for example at least one of mechanical wave emitter 108, 113, energy conductor 109, sound source 111, and waveguide 112. The means 108, 111 are for example an ultrasound transducer or an ultrasound source.

The arrangement can include means for performing localized deposition of the objects 103 contained within a material boundary 104 near bone interface 107, and means for exposing the objects and the bone to the mechanical waves, obtaining deposition 110 of the objects to the bone. The means for performing localized deposition are for example at least one of hollow structure 101, 201, cutting edge 102, reservoir 200 and syringe 203. The means for exposing are for example at least one of the elements represented by reference signs 108, 109, 111, 112 and 113. The means 108, 109, 111, 112, 113 can penetrate skin 105 or tissues 106.

An exemplary arrangement according to the present disclosure can include means for transporting the objects to the bone 107 in order to obtain deposition of the objects 110 to the bone. The means for transporting are for example at least one of means according to reference signs 101, 102, 103, 108 and 109. In an exemplary embodiment the location of weak bone is detected by quantitative ultrasound (QUS) or ultrasound imaging. After defining the weak bone, the deposition of objects into the weak bone can be achieved with mechanical waves generated with the same or a different ultrasound system than used for QUS or ultrasound imaging.

An arrangement according to the present disclosure can include means, such as at least one of means according to reference signs 108, 113, 109, 111, 112 and 204a-e, for generating localized mechanical waves into the tissue to perform the localization on the basis of at least one of high-intensity focused ultrasound (HIFU) 204c, topological guides for ultrasound 112 and electromagnetic steering of the objects to improve focusing of the diffusion. According to exemplary embodiments, it is essential for localized deposition to localize the driving mechanical (or sound) wave field inside the tissue, at for example, a preferred point at or near the bone (e.g. a weak part of the bone). Localization of the driving mechanical wave field can be realized either by means of high-intensity focused ultrasound (HIFU) or topological ultrasound (waveguides or high-order topologies such as fractal structures). Localization can also be realized by means of a counter electrode, or electromagnetic fields that steer the field or objects, to improve focusing of the diffusion.

In an exemplary embodiment according to the present disclosure the arrangement can include means, such as at least one of means according to reference signs 108, 113, 109, 111, 112 and 204a-e, for generating localized mechanical waves into a tissue on the basis of time reversal ultrasound performing adaptive focusing. Time reversal ultrasound permits adaptive focusing through an inhomogeneous medium, such as soft tissue or trabecular bone.

The arrangement can also include means, such as at least one of means according to reference signs 108, 113, 109, 111, 112, 200, 201, 203 and 204a-e, for performing localized deposition of the objects near bone based on photo-acoustic transformation in order to generate mechanical waves near objects. Photo-acoustic transformation also permits introduction of the sound source inside the tissue. In this approach the tissue is irradiated by electromagnetic waves (e.g., laser pulse), which penetrate and absorb into the tissue. The absorption causes localized thermal expansion, which results in emission of mechanical waves (e.g., a sound field) at the point of thermal expansion. The resulting sound field is tunable by parameters of the optical beam (e.g., wavelength, pulse duration, geometric size and shape of the optical beam, number of illuminated spots and/or temporal phasing of an onset of illumination of the different spots). These parameters affect the penetration depth, absorption and scattering in the tissue and determine the emitted sound field. The energy of the electromagnetic beam can be absorbed in any parts of the tissue or in the objects that are being deposited or a combination of thereof. For example, the optical absorption coefficients characteristic to different layers of the soft tissue and bone are functions of the optical wavelength. Thereby, tuning of the optical wavelength permits for example maximization of an absorption ratio between the bone and soft tissue and can result in localization of the sound source at or near the bone. The localization can be also obtained by using a point source (e.g. 108, 113, 204a-b), independent of the technique of implementation.

In another exemplary embodiment according to the present disclosure, the arrangement includes means for selecting the objects from a reservoir of objects and forcing the objects into the bone. Objects (e.g., molecules) are selected from the reservoir of objects (e.g., solution) and are forced into the bone.

The arrangement can include means, such as at least one of means according to reference signs 108, 113, 109, 111, 112, 200, 201, 203 and 204a-e, for performing retention of objects 103, 110 by depositing in addition to the objects a covering layer having a lower perfusion coefficient than the embracing tissues. The purpose of retention is to prevent the deposed objects from escaping the target (bone). Retention is realized by deposing another covering layer that has much lower perfusion coefficient compared to those of the embracing tissues. This can also be realized by depositing a substance that expands and covers the target. An alternative embodiment to this approach is using an active object, which is too large for passive diffusion, but can be actively deposited using the method(s) described herein. The large size can then prevent passive diffusion out of the target. Retention can also be controlled by subsequent sonication: a first application of mechanical waves deposits a substance into the target, followed by several applications of mechanical waves that maintain the substance in the target (counteract the passive diffusion out from the target).

In an exemplary embodiment according to the present disclosure the arrangement can include means, such as at least one of means according to reference signs 101, 102, 103, 108, 113, 109, 111, 112, 200, 201, 203 and 204a-e, for activating objects selectively at different time points. Objects that are inactive in the tissue are first driven in, to form a reservoir 103, 104, 110 of the objects in the tissue. After this, the objects are collectively or selectively activated such as by means of mechanical waves, electromagnetic waves or temperature. Selective activation permits activation at different time points; for example, one ingredient of the objects can be activated directly after drive in and another ingredient can be activated later. This can be considered to be for example catalyzation. In an alternative embodiment, the different drugs are encapsulated in or on a surface of for example, gas voids (with or without lipid shells or equivalent) of various sizes corresponding to various resonant frequencies. After driving in, the encapsulated objects are released at desired moments by sonicating at the resonant frequency corresponding to the release of objects desired.

The arrangement can also include means, such as at least one of means according to reference signs 108, 113, 109, 111, 112, 204a-e, for affecting tissue 107, 105, 106, 110 and 205 by mechanical vibrations. In assembly in situ or in vivo embodiments one, two or several components are driven in the tissue and then treated (shaken) by mechanical vibration. This shaking causes merging of the components to larger aggregates that cannot escape from the target tissue (e.g., bone) or whose escape rate is decreased.

In nanotechnology embodiments according to the present disclosure the arrangement according to the present disclosure can include nanostructure means to control diffusion and to amplify the diffusion. Nano-swimmers or functionalized nano-rods permit improved control of the diffusion and amplification of the diffusion. The same can also be accomplished for example by nano motors, which are controlled by at least one of external field, internal field, external power source and internal power source.

In an exemplary embodiment according to the present disclosure the arrangement can include means (204a-e) for exposing the objects and the bone to the mechanical waves to deposit the objects to the bone utilizing at least one of blood circulation and the bone marrow cavity for the transportation of the objects. Alternatively, instead of driving from the periosteal side of the bone, the objects are driven in bone from the inside (e.g., endosteal side) (means 204b), utilizing blood circulation and/or the bone marrow cavity for the initial transport of the objects to the treatment site, for example, the fracture site, and then exploiting mechanical waves to deposit the objects into the bone.

In exemplary embodiments the arrangement according to the present disclosure can include multi-center-frequency means, such as at least one of means according to reference signs 108, 113, 109, 111, 112 and 204a-e, to generate mechanical waves of at least two different frequencies in order to improve transportation of the objects and deposition of the objects.

Generation of sound waves by at least at two distinct center-frequencies can enhance the drive in. For instance, a kilohertz frequency transducer (e.g., 204d) can be used to increase the permeability at the bone surface (e.g., periosteum or endosteum) and a megahertz frequency transducer (e.g., 204e) can be used to push the objects in.

In exemplary embodiments according to the present disclosure the means, such as at least one of means according to reference signs 108, 113, 109, 111, 112 and 204a-e, for generating localized mechanical waves into a tissue can include a plasma source. Alternatively, instead of using a known ultrasound or photo-acoustic approach, the driving pressure field can be generated by a plasma source. The plasma source can be realized for example, by a focused laser or a spark gap.

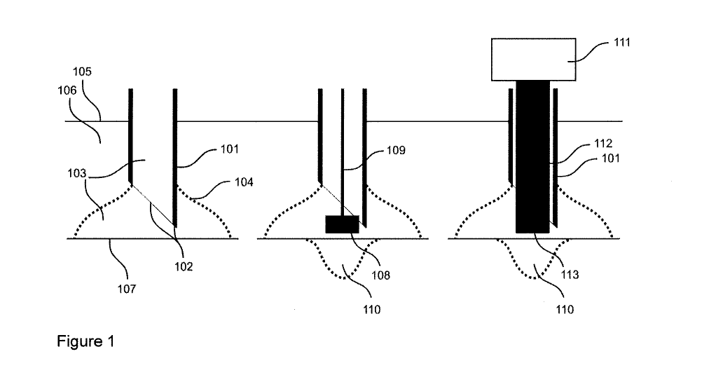

FIG. 1 depicts an exemplary embodiment of the disclosure. A catheter (101) featuring a cutting edge (102) perforates the skin and tissue. The catheter delivers the objects (103) and it forms an object reservoir (103) with boundary 104 near the bone surface (107). The sound source include an energy conductor (109) and a sound emitter (108). The sound emitter 108 may be for example, a flat piezo, a focused piezo, spark, laser induced spark, EMUT (Energy Mode Ultrasound Transducer), CMUT (Capacitive micromachined ultrasonic transducers), PMUT (Piezoelectric Micromachined Ultrasonic Transducers) and equivalent. The mechanical wave generated by the sound emitter translates the objects into the bone (110).

In another exemplary embodiment the sound source (111) is located outside the tissue and the mechanical wave is transmitted to the tissue and active ingredient via a waveguide (112). In an exemplary embodiment of the disclosure, for example, a 10-500 kHz mechanical wave is transmitted through at least one of waveguide (112), active ingredient (103), tissue (106) and sound emitter (108). This mechanical wave alters the permeability of the bone membrane. Another, for example, 0.5-50 MHz, mechanical wave is subsequently transmitted to the boundary. This mechanical wave deposits the active ingredient from the reservoir to the bone.

According to an exemplary embodiment 111 is a light source and a light wave is guided to reservoir 103, 104 and bone 107 through an optical fiber 112 or reflecting inner wall of a catheter 101. The light wave is absorbed by active ingredient 103 or bone 107 to generate light-induced sound waves for translating the active ingredient 103 into the bone 107.

The object can be for example, molecules, drugs, vehicles carrying the object, imaging contrast agent, minerals or nanofibers. Biologically active materials that may be of interest include analgesics, antagonists, anti-inflammatory agents, anthelmintics, antianginal agents, antiarrhythmic agents, antibiotics (including penicillins), anti-cholesterols, anticoagulants, anticonvulsants, antidepressants, antidiabetic agents, antiepileptics, antigonadotropins, antihistamines, antihypertensive agents, antimuscarinic agents, antimycobacterial agents, antineoplastic agents, antipsychotic agents, immunosuppressants, antithyroid agents, antiviral agents, antifungal agents, anxiolytic sedatives (hypnotics and neuroleptics), astringents, beta-adrenoceptor blocking agents, blood products and substitutes, anti-cancer agents, cardiacinotropic agents, contrast media, corticosterioids, cough suppressants (expectorants and mucolytics), diuretics, dopaminergics (antiparkinsonian agents), haemostatics, immunosuppressive and immunoactive agents, lipid regulating agents, muscle relaxants, parasympathomimetics, parathyroid calcitonin and biphosphonates, prostaglandins, radiopharmaceuticals, sex hormones (including steroids), anti-allergic agents, stimulants and anorexics, sympathomimetics, thyroid agents, vasidilators, neuron blocking agents, anticholinergic and cholinomimetic agents, antimuscarinic and muscarinic agents, vitamins, and xanthines. Exemplary medicaments can be e.g. ibandronic acid, zolendronic acid, teriparatide, denosumab, TGF-beta, FGF-beta and BB1/biopharm,

According to an exemplary embodiment, the sound emitter (108) is a confocal transducer featuring two transducers of different center frequencies. According to an exemplary embodiment, the two frequencies generate a third frequency which acts as the wave translating the active ingredient.

According to an exemplary embodiment, catheter wall (101) or waveguide (112) acts as a “cold finger”, such that heat energy is absorbed from the tissues exposed to ultrasound induced heating.

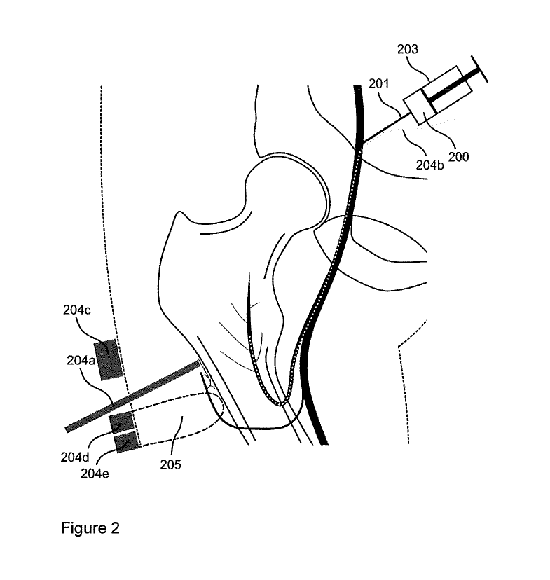

FIG. 2 depicts another means of translating objects, such as active ingredients, into bone. A syringe (201), loaded with the objects containing active ingredient (200), is connected with a needle (201) to a major artery which transports the objects with the blood flow to the treatment site. An ultrasound generator (204a), having at least one of 101, 103, 108, 111, 112, 113, 109 generates the ultrasound which locally translates the drug into the bone. In an alternative embodiment the ultrasound system operates intravenously (204b). In another alternative embodiment, the ultrasound waves are focused through the skin and tissue to the bone-reservoir 103 boundary 104, 107 with an ultrasound generator (204c). In another alternative embodiment, a combination ultrasound system having of two different transducers, one of which (204d) translates the active ingredient 205 through the tissue and skin (such as sono-phoresis), whereas the other one (204e) translates the active ingredient into the bone.

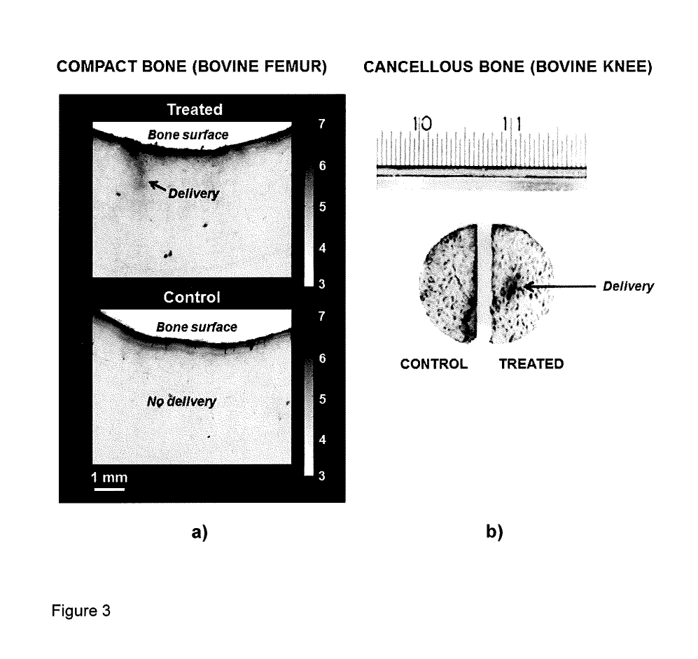

FIG. 3 shows exemplary preliminary results on compact cortical and spongy cancellous bone. (a) Optical microscopy image of cortical bone into which has been delivered contrast agent (methylene blue; image on top) by using high-intensity focused ultrasound (HIU) (Parameters: sine burst frequency: 2.17 MHz; cycles per burst: 200, pulse-repetition frequency: 1000 Hz). The gray scale represents optical absorption. The ultrasound beam enhanced the delivery, as is indicated by an arrow. There is no similar effect seen in a control sample (image on bottom), extracted from the same piece of bone and treated consistently but without ultrasound. (b) Photograph of the result of a related experiment in cancellous bone (sine burst frequency: 2.17 MHz; cycles per burst: 100, pulse-repetition frequency: 600 Hz).

Localized delivery of objects into the bone includes transport and deposition according to the preferred or alternative exemplary embodiments of the disclosure as described in FIGS. 1 and 2. In one phase, one object or a group of objects is transported into the bone. In the second phase, a second object is transported into bone. The second object is delivered close to the pathways through which the first object has travelled to prevent washout of first object. The second object can alternatively self-assemble with itself or with the first object to create large-sized constructs (e.g., via mechanisms such as self-assembly) to slow down or prevent washout of the objects with therapeutic effect. The role of the second object can also be to catalyze the therapeutic effect of first object. The catalyzation can be achieved also by exposing at least one of the objects to mechanical or electromagnetic waves.

Localized deposition is realized by employing one of the presented or different combinations of the presented techniques and methods.

Thus, it will be appreciated by those skilled in the art that the present invention can be embodied in other specific forms without departing from the spirit or essential characteristics thereof. The presently disclosed embodiments are therefore considered in all respects to be illustrative and not restricted. The scope of the invention is indicated by the appended claims rather than the foregoing description and all changes that come within the meaning and range and equivalence thereof are intended to be embraced therein.

1. A kit, comprising
an object delivery arrangement for delivering objects into bone, and retention means configured to counteract the passive diffusion out from the target and formed by one of
a covering layer having a lower perfusion coefficient than the embracing tissues,
an active object having a size sufficient to prevent passive diffusion out of the target in the bone,
a substance that expands and covers the target site in the bone,
subsequent application of mechanical waves for maintaining the substance in the target, after application of mechanical waves for depositing the substance,
said arrangement comprising an ultrasound, photo-acoustics or plasma source configured to generate localized mechanical waves, and
said arrangement being configured to
perform localized deposition of the objects near bone,
expose the objects and the bone to said localized mechanical waves to force the objects into the bone, and
perform retention of the deposited objects in the bone, by using said retention means, so as to prevent the deposed objects from escaping the target site in the bone.

2. A kit according to claim 1, characterised in that the arrangement comprises means for transporting the objects to the bone in order to obtain deposition of the objects into the bone.

3. A kit according to claim 1, characterised in that the arrangement comprises means for generating localized mechanical waves into the tissue performing the localization on the basis of at least one of high-intensity focused ultrasound (HIFU), waveguide, electromagnetic steering of the wave field and electromagnetic steering of the object in order to improve focusing of the diffusion.

4. A kit according to claim 1, characterised in that the arrangement comprises means for generating localized mechanical waves into a tissue on the basis of time reversal ultrasound performing adaptive focusing.

5. A kit according to claim 1, characterised in that the arrangement comprises means for performing localized deposition of the objects near bone on the basis of photo-acoustic transformation in order to localize the means for generating localized mechanical waves inside the tissue and in order to reduce ultrasonic energy deposition in tissue adjacent to bone relative to that in bone.

6. A kit according to claim 1, characterised in that the arrangement comprises means for selecting objects from the reservoir of objects and forcing the objects into the bone tissue.

7. A kit according to claim 1, characterised in that the arrangement comprises means for activating objects selectively at different time points.

8. A kit according to claim 1, characterised in that the arrangement comprises means for affecting a target structure by mechanical vibrations for enhanced deposition.

9. A kit according to claim 1, characterised in that the arrangement comprises nanostructure means to achieve at least one of control diffusion and amplification of the diffusion.

10. A kit according to claim 1, characterised in that the arrangement comprises means for exposing the objects and the bone to said mechanical waves to obtain deposition of the objects to the bone utilizing at least one of blood circulation and the bone marrow cavity for the transportation of the objects.

11. A kit according to claim 1, characterised in that the arrangement comprises multi-center-frequency means to generate mechanical waves of at least two different frequencies in order to improve transportation of the objects and deposition of the objects.
